(19) United States
(12) Patent Application Publication
(10) Pub. No.: US 2023/0103890 A1
(43) Pub. Date: Apr. 6, 2023

(54) Photooxygenation Catalyst Compound and Medicine Comprising Same

(71) Applicant: The University of Tokyo, Tokyo (JP)

(72) Inventors: Youhei Sohma, Tokyo (JP); Motomu Kanai, Tokyo (JP); Nozomu Nagashima, Tokyo (JP); Taisuke Tomita, Tokyo (JP); Yukiko Hori, Tokyo (JP); Shuta Ozawa, Tokyo (JP)

(21) Appl. No.: 17/780,720

(22) PCT Filed: Dec. 2, 2020
(86) PCT No.: PCT/JP2020/044846

§ 371 Date: Aug. 10, 2022

(30) Foreign Application Priority Data
Dec. 2, 2019 (JP) 2019-217947

(51) Int. Cl. A61K 41/00; B01J 31/02; B01J 35/00; C07F 5/02; A61P 25/28

(52) U.S. Cl. CPC A61K 41/0057; B01J 31/0275; B01J 35/004; C07F 5/027; A61P 25/28; B01J 2531/002; B01J 2231/70

(57) ABSTRACT

[Problem] The present invention addresses the problem of developing a catalyst compound which has blood-brain barrier penetration properties and enables the oxygenation of amyloids in a body upon being irradiated with light from the outside of the body and providing a prophylactic and therapeutic agent for amyloid-related diseases using the catalyst compound.

[Solution] It is found that a compound having such a framework that an azobenzene-like structure and boron together form a complex is useful as a novel biocatalyst which can selectively oxygenate an amyloid and can prevent the aggregation of the amyloid upon being irradiated with light while significantly reducing the molecular weight of the amyloid. It is also found that the compound can exhibit an oxygenation activity upon the irradiation with light having a longer wavelength which has high tissue penetration properties and has excellent blood-brain barrier penetration properties.

### THE TECHNOLOGY FIELD

The present invention relates to pharmaceuticals for preventing or treating diseases associated with various pathogenic amyloids.

### BACKGROUNDS

In general, proteins form a specific native structure by folding and carry out vital functions, but on the other hand, by misfolding, they aggregate (amyloidized) into fibers rich in (β-sheet structure. Aggregates (oligomers, protofibrils, fibers) produced in the process of this amyloidization are known to cause various dysfunctions (such diseases are collectively referred to as “amyloid disease”). More than 35 types of protein have been identified as the causative agent of amyloid disease. Known examples of such amyloid protein include tau for Alzheimer's disease, α-synuclein for Parkinson's disease, amylin for diabetes, transthyretin for systemic amyloidosis, and huntingtin for Huntington's disease.

On the other hand, Alzheimer's disease is a progressive neurodegenerative disease that causes cognitive decline along with brain atrophy, and the number of patients is increasing year by year. This Alzheimer's disease is also a type of amyloid disease, and its onset is thought to involve neurotoxicity due to aggregates formed by amyloid β (Aβ). As therapeutic agents and therapeutic methods targeting Aβ, for example, inhibitors of enzymes that produce Aβ from precursor proteins, Aβ-degrading enzyme promoters, immunotherapy, Aβ aggregation inhibitors and the like are known. However, these conventional treatment methods have problems such as side effects and low pharmacological effect, and have not yet been put into practical use. Therefore, it is desired to develop a new method leading to safe and effective treatment of Alzheimer's disease.

On the other hand, the inventors of the present application have developed a compound capable of reducing the aggregation and toxicity of Aβ by a photooxygenation reaction that imparts an oxygen atom to Aβ (Non-Patent Documents 1 to 3), etc). However, since these compounds have a large molecular weight, they have low blood-brain barrier permeability, and there are practical problems such as the need to administer the compounds into the brain by an invasive method such as surgery.

### PRIOR ARTS

### Non-Patented References

Reference 1: Taniguchi, A. et al., Nat. Chem. 2016, 8, 974-982

Reference 2: Ni, J. et al., M. Chem 2018, 4, 807-820

Reference 3: Suzuki, T. et al., Chem. Commun. 2019, 55, 6165

### SUMMARY OF INVENTION

### Problems to be Solved by Invention

In view of the problems of the prior art, the present invention develops and uses a catalyst compound which has blood-brain barrier permeability and enables oxygenation of amyloid in the body by irradiation with light from outside the body. The subject is to provide a preventive and therapeutic drug for amyloid-related diseases.

### Means to Solve Problems

As a result of diligent studies to solve the above problems, the present inventors have found that a compound having a structure in which an azobenzene-like structure and boron are complexly formed is useful as a novel in vivo catalyst that selectively oxygenates amyloids and suppresses aggregation while significantly reducing the molecular weight. It was found that the compound exhibits oxygenation activity by irradiation of the long wavelength light with high tissue permeability and has excellent permeability to the blood-brain barrier. These findings have led to the completion of the present invention. Here, “oxygenation”, one of the oxidation reactions, means a reaction in which an oxygen atom is bound.

That is, the present invention, in one aspect,

<1> A compound represented by the following formula (Ia) or (Ib) or its salt thereof:

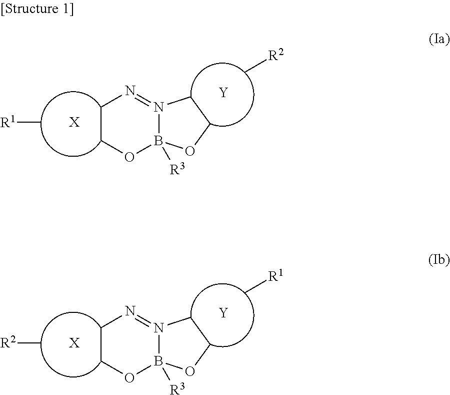

(wherein X and Y are aromatic rings that may be independently the same or different; R¹ is a substituent selected from the group consisting of an amino group, an alkyl group, an alkoxy group, a sulfo group, a phosphoric acid group, and a heteroaryl groups with a hydrophilic substituent, which may be optionally substituted, and which may be present at any position on X or Y; R² is a halogen atom, a selenium atom, or a haloalkyl group having 1-3 carbon atoms, which may be present at any position on Y or X; and R³ is a halogen atom or a haloalkyl group having 1-3 carbon atoms);

<2> The compound or salt thereof according to <1>above, wherein X and Y are benzene rings or naphthalene rings;

<3> The compound or salt thereof according to the above <1> represented by the following formula (IIa) or (IIb):

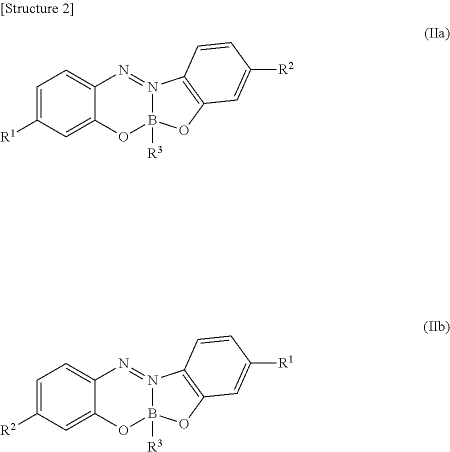

(wherein R¹, R², and R³ are the same as the definition in claim 1).

<4> The compound or salt thereof according to any one of <1> to <3> above, wherein R² is a bromine atom, an iodine atom, or a selenium atom;

<5> The compound or salt thereof according to any one of <1> to <4> above, wherein R³ is a fluorine atom or a fluoroalkyl group having 1 to 3 carbon atoms.

<6> The compound or salt thereof according to any one of <1> to <5>, wherein R¹ is represented by the following formula (a):

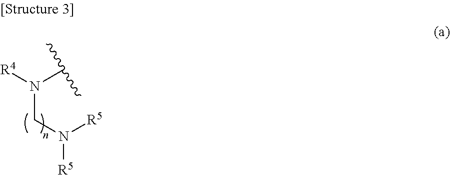

(In the formula, the broken line indicates the connection to X or Y; R⁴ is a hydrogen atom, an alkyl group or an aromatic ring which may be optionally substituted; R⁵ is a hydrogen atom, an alkyl group or an aromatic ring, which may be optionally substituted, which may be independently the same or different.; n is an integer of 0 to 5.);

<7> A compound selected from the following groups or salt thereof: and

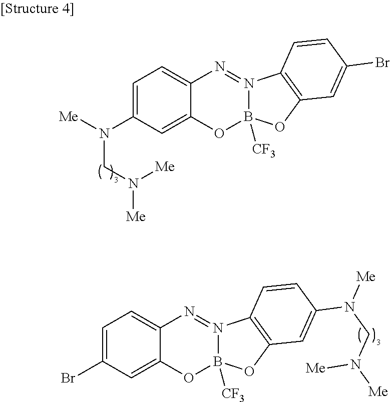

(wherein Me represents a methyl group.)

In another aspect, the present invention also relates to a pharmaceutical and a therapeutic method containing the above-mentioned compound, and more specifically, the present invention relates to the above-mentioned compound.

<8> An oxygenation catalyst for pathogenic amyloids containing the compound according to any one of <1> to <7> or salt thereof;

<9> A pathogenic amyloids aggregation inhibitor containing the compound according to any one of <1> to <7> or salt thereof;

<10> A pharmaceutical composition containing the compound according to any one of <1> to <7> or salt thereof and a pharmaceutically acceptable carrier;

<11> The pharmaceutical composition according to <10> above, which is a prophylactic or therapeutic agent for a disease associated with pathogenic amyloids;

<12> The pharmaceutical composition according to <11> above, wherein the disease associated with the pathogenic amyloids is Alzheimer's disease;

<13> Use of the compound according to any one of <1> to <7> or salt thereof in producing an agent for prevention or treatment of a disease associated with pathogenic amyloids.

<14> A method for preventing or treating a disease associated with pathogenic amyloids, which comprises administering an effective amount of the compound according to any one of <1> to <7> or its salt thereof

<15> The method according to <14> above, which comprises irradiating the affected area of the patient with light from outside the body after administration of the compound according to any one of <1> to <7> or salt thereof;

<16> The method according to <14> or <15>, wherein the disease associated with the pathogenic amyloids is Alzheimer's disease.

### The Effect of Invention

The present invention provides the photooxygenation catalyst compounds which has high activity for oxygenating pathogenic amyloids such as Aβ peptide by irradiation the long wavelength light having high tissue permeability, and has excellent permeability to the blood-brain barrier. This makes it possible to suppress or reduce the aggregation and toxicity of pathogenic amyloids in vivo (in the brain, etc.) by a non-invasive method of irradiating light from outside the body after administration by intravenous administration or the like. Therefore, the present invention enables prevention and treatment of diseases associated with pathogenic amyloids by a minimally invasive method that has never existed in the past.

### A BRIEF DESCRIPTION OF THE FIGURES

FIG. 1 is a spectrum of MALDI-TOF-MS in the photooxygenation reaction of Aβ by the compound of the present invention.

FIG. 2 is a graph showing the amount of oxygenated Aβ produced in the photooxygenation reaction of Aβ by the compound of the present invention.

FIG. 3 is a graph showing peptide selectivity in a photooxygenation reaction with a compound of the present invention.

FIG. 4 is a graph showing a comparison of the amount of oxygenated Aβ produced in Compound 2 and Comparative Example 1 in the Aβ oxygenation reaction.

FIG. 5 shows (A) changes in the fluorescence spectra of compound 2 in the presence and absence of aggregated Aβ; (B) the most stable structure of compound 2 in the ground and excited states optimized using the density functional theory.

FIG. 6 shows the oxygenation site in the photooxygenation reaction of Aβ by the compound of the present invention.

FIG. 7 is a graph showing the inhibition of aggregation of Aβ in the photooxygenation reaction by the compound of the present invention.

FIG. 8 shows the blood-brain barrier (BBB) permeability of the compounds of the present invention and Comparative Examples (Compounds 3 and 4).

FIG. 9 shows the results of the photooxygenation reaction of Aβ by the compound of the present invention in Alzheimer's disease model mice.

### EMBODIMENT FOR CARRYING OUT THE INVENTION

Hereinafter, embodiments of the present invention will be described. The scope of the present invention is not limited to these explanations, and other than the following examples, the present invention can be appropriately modified and implemented without impairing the gist of the present invention.

### 1. Definition

As used herein, the term “halogen atom” means a fluorine atom, a chlorine atom, a bromine atom, or an iodine atom.

In the present specification, the “alkyl or alkyl group” may be any of a linear, branched, cyclic, or a combination thereof, an aliphatic hydrocarbon group. The number of carbon atoms of the alkyl group is not particularly limited, but is, for example, 1 to 20 carbon atoms (C1 to C20), 1 to 15 carbon atoms (C1 to C15), and 1 to 10 carbon atoms (C1 to C10). As used herein, the alkyl group may have one or more arbitrary substituents. For example, C1-8 alkyl includes methyl, ethyl, n-propyl, isopropyl, n-butyl, isobutyl, sec-butyl, tert-butyl, n-pentyl, isopentyl, neo-pentyl, n-hexyl, iso-hexyl, n-heptyl, n-octyl and the like are included. Examples of the substituent include an alkoxy group, a halogen atom (which may be a fluorine atom, a chlorine atom, a bromine atom, or an iodine atom), an amino group, a mono or di-substituted amino group, a substituted silyl group, or a substituted acyl and the like can be mentioned, but the present invention is not limited to these. If the alkyl groups have more than one substituent, they may be the same or different. The same applies to the alkyl moiety of other substituents containing the alkyl moiety (e.g., an alcoholic group, an arylalkyl group, etc.).

In the present specification, the “alkoxy group” is a structure in which the alkyl group is bonded to an oxygen atom, and examples thereof include saturated alkoxy groups which are linear, branched, cyclic, or a combination thereof. For example, methoxy group, ethoxy group, n-propoxy group, iso-propoxy group, cyclopropoxy group, n-butoxy group, isobutoxy group, s-butoxy group, t-butoxy group, cyclobutoxy group, cyclopropylmethoxy group, n-Pentyloxy group, cyclopentyloxy group, cyclopropylethyloxy group, cyclobutylmethyloxy group, n-hexyloxy group, a cyclohexyloxy group, a cyclopropylpropyloxy group, a cyclobutylethyloxy group, a cyclopentylmethyloxy group and the like are preferable examples.

In the present specification, the “aromatic ring” means a monocyclic or fused polycyclic conjugated unsaturated hydrocarbon ring structure, and a hetero atoms (for example, an oxygen atom, a nitrogen atom, or a sulfur atom, etc.) may be contained in one or more as a ring-constituting atom.

In the present specification, the “aryl or aryl group” may be either a monocyclic or condensed polycyclic aromatic hydrocarbon group, and the heteroatom as a ring-constituting atom may be used. It may be an aromatic heterocycle containing one or more atoms (such as an atom, a nitrogen atom, or a sulfur atom). In this case, this is referred to as a “heteroaryl group” or a “heteroaromatic group”. Whether the aryl is a monocyclic ring or a condensed ring, it can be bonded at all possible positions. Non-limiting examples of monocyclic aryls include phenyl group (Phe), thienyl group (2- or 3-thienyl group), Pyridyl group, frill group, thiazolyl group, oxazolyl group, pyrazolyl group, 2-pyrazinyl group, Pyrimidinyl group, pyrrolyl group, imidazolyl group, pyridazinyl group, 3-isothiazolyl group, 3-isooxazolyl group, 1,2,4-Examples thereof include an oxadiazole-5-yl group or a 1,2,4-oxadiazole-3-yl group. Non-limiting examples of fused polycyclic aryls include 1-naphthyl group, 2-naphthyl group, 1-indenyl group, 2-indenyl group, 2,3-dihydroinden-1-yl group, 2,3-dihydroinden-2-yl group, 2-anthril group, indazolyl group, quinolyl group, isoquinolyl group, 1,2-dihydroisoquinolyl group, 1,2,3,4-tetrahydroisoquinolyl group, indolyl group, isoindryl group, phthalazinyl group, Quinoxalinyl group, benzofuranyl group, 2,3-dihydrobenzofuran-1-yl group, 2,3-dihydrobenzofuran-2-yl group, naphthilidinyl, dihydronaphthyldinyl, tetrahydronaphthyldinyl, imidazolipyridinyl, pteridinyl, prynyl, Kinolidinyl, Indridinyl, Tetrahydroquinolidinyl, and Tetrahydroindolidinyl, 2,3 -dihydrobenzothiophen-1-yl group, 2,3-dihydrobenzothiophen-2-yl group, benzothiazolyl group, benzimidazolyl group, fluorenyl Examples include a group or a thioxanthenyl group. As used herein, an aryl group may have one or more arbitrary substituents on its ring. Examples of the substituent include, but are not limited to, an alkoxy group, a halogen atom, an amino group, a mono or di-substituted amino group, a substituted silyl group, and an acyl. If the aryl group has two or more substituents, they may be the same but different. The same applies to the aryl moiety of other substituents containing the aryl moiety (eg, aryloxy group, arylalkyl group, etc.).

However, it is not limited to these. If the aryl group has two or more substituents, they may be the same or different.

In the present specification, “alkylamino” and “arylamino” mean an amino group in which the hydrogen atom of —NH₂ group is substituted with 1 or 2 of the above alkyl or aryl. For example, methylamino, dimethylamino, ethylamino, diethylamino, ethylmethylamino, benzylamino and the like can be mentioned.

When a functional group is defined as “may be substituted” in the present specification, the type of substituent, the position of substitution, and the number of substituents are not particularly limited. If they have more than one substituent, they may be the same or different. Examples of the substituent include, but are not limited to, an alkyl group, an alkoxy group, a hydroxyl group, a carboxyl group, a halogen atom, a sulfo group, an amino group, an alkoxycarbonyl group, an oxo group and the like. Further substituents may be present in these substituents. Examples of such include, but are not limited to, alkyl halide groups.

### 2. The Compound of the Present Invention

The compound of the present invention has a structure in which an azobenzene-like structure and boron are complexly formed, and is represented by the following formula (Ia) or (Ib).

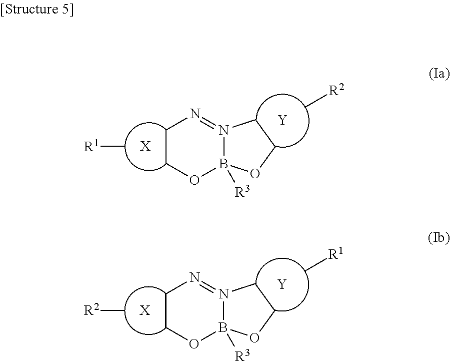

The difference between the formula (Ia) and (Ib) is that in the formula (Ia), R1 is connected to X and R2 is connected to Y, whereas in the formula (Ib), R2 is connected to X and R1 is connected to Y

In the equation, X and Y are aromatic rings that may be independently the same or different from each other. X and Y are preferably a benzene ring or a naphthalene ring, and more preferably a benzene ring.

When both X and Y are benzene rings, the compound of the present invention has a structure in which azobenzene and boron are complexly formed, and is represented by the following formula (IIa) or (IIb) in a preferred embodiment.

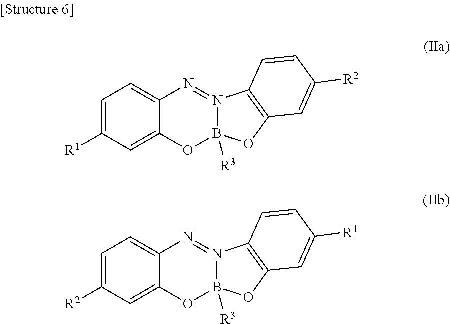

In any of the formulas (Ia), (Ib), (IIa), and (IIb), R¹ is a substituent selected from the group consisting of an amino group, an alkyl group, an alkoxy group, a sulfo group, a phosphoric acid group, and a heteroaryl groups with a hydrophilic substituent, which may be optionally substituted respectively. The R1 is preferably electron-donating and hydrophilic in order to enhance the solubility of the compound of the present invention. The alkyl group and the alkoxy group are preferably C1 to C10 of a straight chain or a branched chain, and more preferably C1 to C5 of a straight chain or a branched chain. Further, the heteroaryl group in the “heteroaryl group having a hydrophilic substituent” can be, for example, thiophene or selenophene; and the hydrophilic substituent can be, for example, an amino group or a carboxyl group.

In the case of the formula (Ia), R1 may exist at an arbitrary position on X, and in the case of the formula (Ib), R1 may exist at an arbitrary position on Y. Preferably, R1 can be linked to X or Y at the meta position with respect to the N atom of the azo group, as shown in formulas (IIa) and (IIb).

In a more preferred embodiment, R1 can be an amino group represented by the following formula (a).

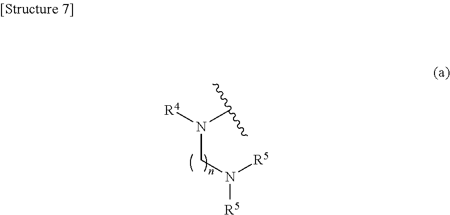

In formula (a), the broken line indicates the connection to X or Y; R4 is a hydrogen atom, an alkyl group or an aromatic ring which may have a substituent, and R5 is independently the same or different, and may be a hydrogen atom, an alkyl group or an aromatic ring which may have a substituent; n is an integer of 0 to 5. Preferably, R4 and R5 are linear or branched C1-C5 alkyl groups that may be independently the same or different, and more preferably both are methyl groups. Further, preferably, n is an integer of 1 to 3.

R2 is a group capable of producing a heavy atom effect, and in any of the formulas (Ia), (Ib), (IIa) and (IIb), a halogen atom, a selenium atom or a haloalkyl having 1 to 3 carbon atoms. R2 is preferably a bromine atom, an iodine atom, or a selenium atom; or an alkyl bromide or an alkyl iodide of C1 to C3.

In the case of the formula (Ia), R2 may be present at an arbitrary position on Y, and in the case of the formula (Ib), R2 may be present at an arbitrary position on X. Preferably, R2 can be linked to X or Y at the meta position with respect to the N atom of the azo group, as shown in formulas (IIa) and (IIb).

R3 is a halogen atom or a haloalkyl group having 1 to 3 carbon atoms in any of the formulas (Ia), (Ib), (IIa) and (IIb). R3 is preferably a group exhibiting strong electron-withdrawing properties. Typically, R3 is preferably a fluorine atom or a fluoroalkyl group having 1 to 3 carbon atoms. The haloalkyl group may be a straight chain or a branched chain.

Specific examples of the compound of the present invention include compounds having the following structures (Me represents a methyl group in any of the formulas). However, it is not limited to these.

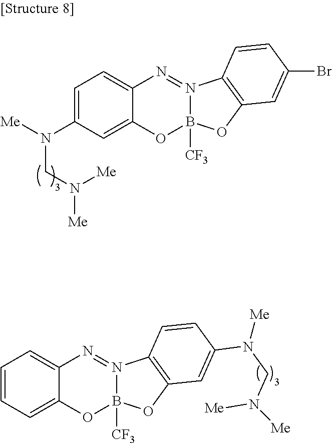

The compound of the present invention represented by the above formulas (Ia) and (Ib) may exist as a salt. Examples of such salts include base-added salts, acid-added salts, amino acid salts and the like. Examples of the base addition salt include metal salts such as sodium salt, potassium salt, calcium salt and magnesium salt, ammonium salt, and organic amine salts such as triethylamine salt, piperidine salt and morpholin salt, and acid addition salt. Examples thereof include mineral salts such as hydrochlorides, sulfates and nitrates, and organic acid salts such as carboxylates, methanesulfonates, paratoluenesulfonates, citrates and oxalates . . .

Examples of the amino acid salt include a glycine salt and the like. However, it is not limited to these salts.

The compound of the present invention may have one or more asymmetric carbons, depending on the type of substituent, and may have stereoisomers such as optical isomers or diastereoisomers. be. Pure forms of steric isomers, arbitrary mixtures of steric isomers, racemates and the like are all included within the scope of the invention.

Further, the compound of the present invention or its salt thereof may exist as a hydrate or a solvate, and all of these substances are included in the scope of the present invention. The type of solvent that forms the solvate is not particularly limited, and examples thereof include solvents such as water, ethanol, acetone, and isopropanol.

The embodiments of the present specification specifically show a method for producing a representative compound included in the compound of the present invention, and those skilled in the art can refer to the disclosure of the present specification. , And, if necessary, starting materials, reagents, reaction conditions and the like can be appropriately selected to easily produce any compound included in the formulas (Ia) and (Ib). Typically, as shown in Examples, the present invention is obtained by reacting a compound having an azobenzene-like structure as a starting material with a halogenated boric acid compound, and then appropriately introducing a substituent corresponding to R1 or R2. Compounds can be obtained.

### 2. Pharmaceutical Compositions Containing the Compounds of the Present Invention, Etc.

The compounds of the present invention can catalyze the oxygenation reaction of pathogenic amyloids. The oxygenation reaction proceeds by adding an oxygen atom to an amino acid residue in the amyloid and oxidizing the compound of the present invention in an excited state by irradiation with light. This makes it possible to suppress or reduce the aggregation of pathogenic amyloids.

Accordingly, the present invention relates to, in another embodiment, an oxygenation catalyst for primary amyloid or an inhibitor for aggregation of pathogenic amyloids, which comprises the above compound or its salt thereof. Furthermore, the present invention also relates to a pharmaceutical composition containing the above compound or its salt thereof and a pharmaceutically acceptable carrier.

The compound of the present invention is particularly characterized by excellent oxygenation activity against aggregated pathogenic amyloids. Although not necessarily bound by theory, as shown in the above formulas (Ia) and (Ib), it has a structure in which an azobenzene-like structure and boron are complexly formed, the molecular structure is bent and the excited state is relaxed when it is irradiated with excitation light. On the other hand, in an environment where the molecules are densely bound to aggregated amyloid, the change in the molecular structure is suppressed and singlet oxygen is generated, so that the amyloid can be selectively oxygenated.

“Pathogenic amyloids” includes amyloid β (Aβ) peptides and amyloids, which are known to be involved in Alzheimer's disease, Parkinson disease, diabetes, Huntington's disease, and systemic amyloidosis in animals including humans. Also, including amyloid such as Amylin, Transthyretin, α-synuclein, tau protein, Huntingtin and other amyloids. However, it is not limited to these.

For example, when the Aβ peptide is oxidized by the compound of the present invention, one or more amino acid residues among the 40 or 42 amino acid residues constituting the Aβ peptide may be oxidized, but it is selected from His and Met. It is preferable that one or more amino acid residues are oxidized. The oxidation is more preferably in the form of adding a hydroxy group or an oxo group (oxide) to each amino acid residue. In the case of His, it is presumed that the oxidant of the amino acid residue has a structure in which the imidazole ring of the histidine residue is oxidized, that is, a dehydro-imidazolone ring and a hydroxy-imidazolone ring. In the case of Met, it is presumed that oxygen is added to the sulfur atom in the methionine residue.

The compound of the present invention preferably has a maximum absorption wavelength (λmax) in the range of 550 to 800 nm, and is preferably excited at that wavelength. By having an absorption band on the long wavelength side, it can be excited by long wavelength light having high bio-permeability.

Further, the compound of the present invention preferably has a molecular weight of 300 to 550. By having such a relatively small molecular weight, it is possible to exhibit excellent permeability to the blood-brain barrier.

The pharmaceutical composition containing the compound of the present invention or its salt thereof can be prepared by a preparation method of various preparations by selecting an appropriate preparation according to the administration method and using a pharmaceutically acceptable carrier. Examples of the dosage form of the pharmaceutical composition containing the compound of the present invention as a main agent include tablets, powders, granules, capsules, liquids, syrups, elixirs, oily or aqueous suspensions and the like as oral preparations.

As an injection, a stabilizer, a preservative, or a solubilizing agent may be used in the preparation, and a solution containing these auxiliary agents may be stored in a container and then freeze-dried to be used as a solid preparation. It may be a time-prepared preparation. Further, a single dose may be stored in one container, or a large dose may be stored in one container.

Examples of the external preparation include liquids, suspensions, emulsions, ointments, gels, creams, lotions, sprays, patches and the like.

The solid preparation contains a pharmaceutically acceptable additive together with the compound of the present invention, for example, fillers, bulking agents, binders, disintegrants, dissolution accelerators, wetting agents, and lubricants. Agents and the like can be selected, mixed and formulated as needed. Examples of the liquid preparation include solutions, suspensions, emulsions and the like, but the additives may include suspending agents, emulsifiers and the like.

When the compound of the present invention is used as a pharmaceutical for the human body, the dose is preferably in the range of 1 mg to 1 g, preferably 1 mg to 300 mg per day for an adult.

In a further aspect, the invention also relates to a method of preventing or treating a disease associated with pathogenic amyloids, which comprises administering an effective amount of the compound or its salt thereof. The method preferably comprises irradiating the affected area of the patient with light from outside the body after administration of the compound or salt thereof. As described above, since the compound of the present invention can be excited by long-wavelength light having high bio-permeability, a non-invasive method of irradiating light from outside the patient's body after administration by intravenous administration or the like is used. Aggregation and toxicity of pathogenic amyloids in vivo (brain, etc.)

It can be suppressed or reduced.

Specifically, the compound of the present invention or its salt thereof may be introduced into a living body or a cell, and irradiated with light when the compound is transferred to a target site. Examples of the means for administration into the living body include intramuscular injection, intravenous injection, local administration, oral administration and the like.

Examples of diseases associated with pathogenic amyloids include Alzheimer's disease, Parkinson's disease, diabetes, Huntington's disease, systemic amyloid-cis and the like in animals including humans. Typically, the disease associated with pathogenic amyloids is Alzheimer's disease.

### WORKING EXAMPLES

Hereinafter, the present invention will be described in more detail by way of examples, but the present invention is not limited thereto.

### 1. Synthesis of the Compound of the Present Invention

The compound of the present invention (“Compound 2”) was synthesized by the following procedure.

[Synthesis of Compound 1]:

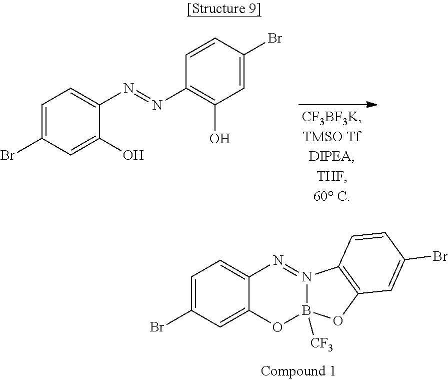

To a solution of (E)-6,6′-(diazen-1,2-diyl) bis (3-bromophenol) (30 mg, 0.081 mmol, 1.0 eq) in THF (3.0 mL). CF3BF3K (57 mg, 0.32 mmol, 4.0 eq), TMSOTf (97.2 μL, 0.53 mmol, 6.7 eq), and DIPEA (68.8 μL, 0.40 mmol, 5.0 eq) were added and the mixture was stirred at 60° C. overnight. After cooling, the mixture was concentrated and dissolved in CH2Cl2 and H2O. Extract the mixture with CH2Cl2 (3 times), and the organic layer was washed with saline, dried over Na2SO4 and concentrated. The obtained residue was purified by silica column chromatography (hexane/CH2Cl2=3/1) to give compound 1 as a red solid (16.8 mg, 0.037 mmol, 46% yield).

1H NMR (CDCl3, 500 MHz): δ=7.70 (d, J=4.5 Hz, 1H), 7.66 (d, J=8.6) Hz, 1H), 7.42 (m, 2H), 7.33 (m, 1H), 7.26 (m, 1H); 13C NMR (CDCl3, 126 MHz): δ=162.1, 144.8, 139.3, 134,5, 132.7, 132.5, 132.4, 126.7, 126.3, 123.2, 119.9, 117.7; 11B NMR (CDCl3, 126 MHz): δ=0.30, 19F NMR (CDCl3, 369 MHz): δ=−73.6.

[Synthesis of Compound 2]:

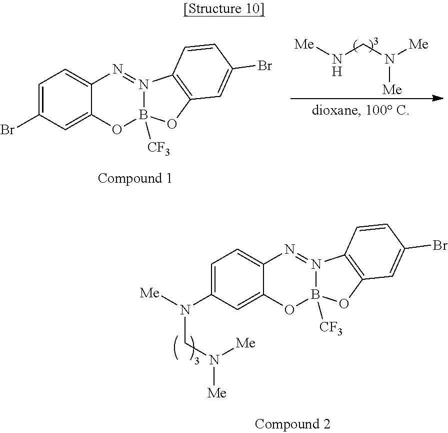

N, N, N′-trimethyl-1,3-propanediamine (5.6 μL, 0.038 mmol, 1.0 eq) was added to a 1,4-dioxane (0.50 mL) solution of compound 1 (17 mg, 0.038 mmol, 1.0 eq). and the mixture was stirred at 100° C. for 1 hour. After the reaction, the mixture was concentrated and dissolved in MeCN. Compound 2 was purified by HPLC and obtained as a purple solid (13.1 mg as TFA salt, 0.0219 mmol, yield 58%).

¹H NMR (CDCl3, 400 MHz): δ=7.54 (d, J=9.6 Hz, 1H), 7.45 (d, J=8.7 Hz, 1H), 7.25 (d, J=1.4 Hz, 1H), 7.12 (dd, J=8.7 Hz, 1.4 Hz, 1H), 6.59 (dd, J=9.6 Hz, 2.7 Hz, 1H), 6.25 (d, J=2.7 Hz, 1H), 3.65 (m, 2H), 3.21 (s, 3H)), 3.09 (t, J=8.2 Hz, 2H), 2.84 (s, 6H), 2.17 (quin, J=8.2 Hz, 2H); 13C NMR (CDCl3, 99 MHz): δ=158.1, 157.7, 149.5, 135.2, 135.1, 133.4, 125.4, 124.5, 118.1, 115.5, 109.6, 98.9, 54.8, 49.9, 43.0, 39.1, 23.0; 11B NMR (CDCl3, 126 MHz): δ=0.31, 19F NMR (CDCl3, 369 MHz):=δ−74.6 (3F), −75.1 (3F); [M+H]+485.1, found 484.9

Further, as a comparative example, a compound having no bromine atom at the position of R2 of compound 2 was synthesized.

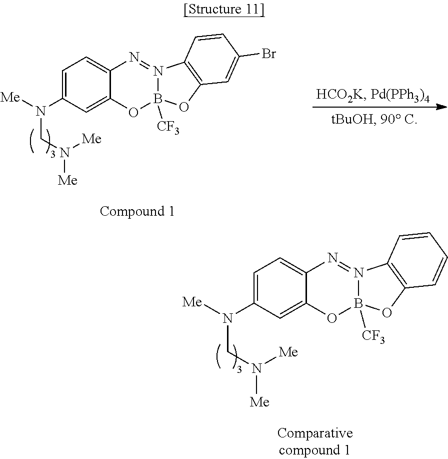

HCO2K (6.8 mg, 0.081 mmol, 5.0 eq) and Pd (PPh3) 4 (5.7 mg, 0.0049 mmol, 0.3 eq) were added to a tBuOH (1.0 mL) solution of compound 2 (7.9 mg, 0.016 mmol, 1.0 eq), and the solution was frozen and degassed three times. The mixture was then heated at 90° C. for 1 hour. The volatiles were removed under reduced pressure. The obtained residue was dissolved in MeCN and purified by HPLC to obtain comparative example compound 1 as a purple solid (1.5 mg as TFA salt, 0.0029 mmol, yield 18%).

¹H NMR (CD3CN, 500 MHz): δ=7.58-7.54 (m, 2H), 7.31 (dt, J=8.0 Hz, 1.1 Hz, 1H), 7.03 (m, 2H), 6.78 (dd, J=9.2 Hz, 2.3 Hz, 1H), 6.39 (d, J=2.3 Hz, 1H), 3.66-3.59 (m, 2H), 3.20 (s, 3H), 3.07 (m, 2H), 2.76 (s, 6H), 2.06 (m, 2H); ¹³C NMR (CDCl₃, 126 MHz): δ=158.2, 157.3, 149.3, 135.0, 134.8, 134.2, 132.4, 121.2, 114.9, 114.8, 109.1, 98.9, 55.0, 49.8, 43.1, 39.0, 23.1; ¹¹B NMR (CDCl3, 126 MHz): δ=−0.31, ¹⁹F NMR (CDCl₃, 369 MHz): δ=−74.4 (3F), −75.2 (3F); LRMS (ESI): m/z calculated for [M+H]+407.2, found 407.0

### 2. Oxidation of amyloid β (Aβ)

Firstly, Aβ1-42 was prepared in situ from 26-O-acyl isopeptide (commercially available from Peptide Institute, Inc.). References (Taniguchi, A.; Sasaki, D.; Shiohara, A.; Iwatsubo, T.; Tomita, T.; Sohma, Y.; Kanai, M. Angew. Chem. Int. Ed. 2014, 53 , 1382).

According to the description in the above references, Aβ1-42 isopeptide (200 μM in 0.1% trifluoroacetic acid aqueous solution), angiotensin IV (200 μM in water), [Tyr8]-Substance P (200 μM in water), leuprorelin acetate. (200 μM in water), somatostatin (200 μM in water) was diluted with 100 mM phosphate buffer or phosphate buffered saline (PBS; pH 7.4) to a final peptide concentration of 20 μM (pH 7.4). For Aβ1-42, the solution was incubated at 37° C. for 3 hours.

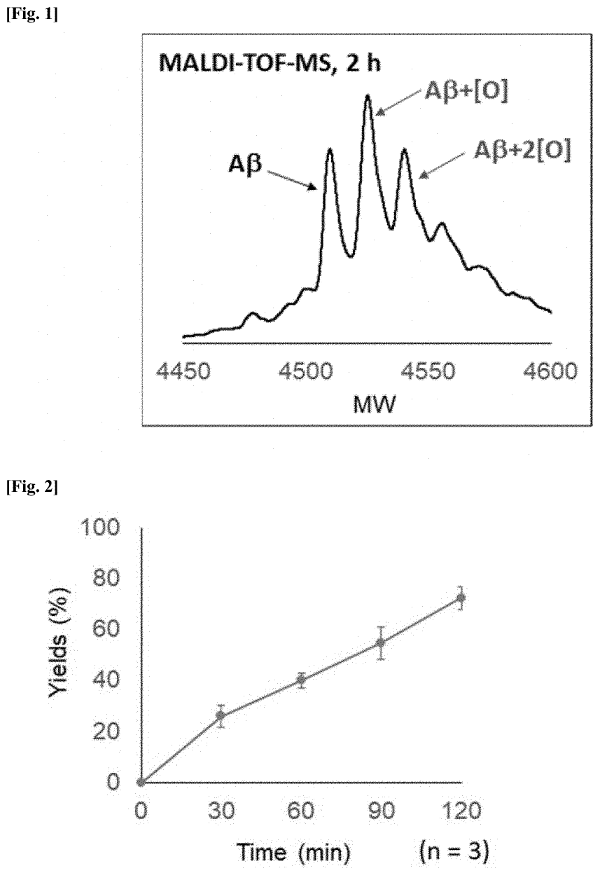

Compound 2 or comparative example compound 1 (2 mM in dimethyl sulfoxide) was added to each solution. A mixture containing compound 2 (or comparative example 1) having a final concentration of 40 μM was irradiated with a light emitting diode (LED) (λ=595 nm) at 37° C. The output of the 595 nm LED light source was 10 mW, and the light irradiation was performed at a distance of about 5 cm from the sample. Corresponding reaction samples without light irradiation were also prepared as controls. Reactions were monitored and analyzed using MALDI-TOF MS. If necessary, the reaction solution was desalted with ZipTip U-C18 (Millipore Corporation) prior to MS analysis. The degree of oxygenation is shown as the oxygenation intensity ratio (%)=(total of MS peak intensities of n [O] adduct)/(total of MS peak intensities of remaining starting material and n [O] adduct)×100. FIG. 1 shows the spectrum of the obtained MALDI-TOF-MS. FIG. 2 shows the amount of oxygenated Aβ produced.

As shown in FIG. 2, it was found that the oxygenation reaction of aggregated Aβ proceeded smoothly by irradiating with light of 595 nm. In addition, FIG. 3 shows the results of evaluation of selectivity using off-target peptides having tryptophan, tyrosine, histidine, methionine and the like. As a result, under the condition that the reaction of Aβ proceeded at a yield of 73%, the yield of other peptides remained at 4% or less, and therefore the compound 2 promoted the oxygenation reaction of Aβ with high selectivity.

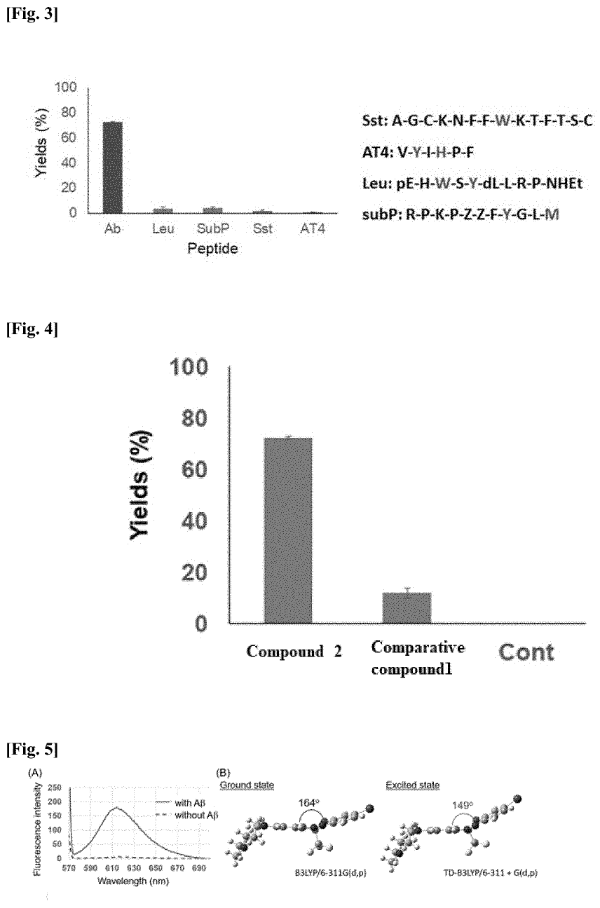

Further, FIG. 4 shows a comparison between compound 2 and comparative example 1 in the Aβ oxygenation reaction.

As a result, it was found that the compound 2 having a bromine atom at the position of R2 in the formula (Ia) exhibits significantly higher oxygenation activity than that of comparative example 1 in which the position of R2 is a hydrogen atom. Note that Cont in the figure is the result of irradiating only light without adding a catalyst.

### 3. Oxygenation of Other Amyloids

The photooxygenation of amylin, α-synuclein, and insulin, which are amyloids having a cross β sheet structure, was performed in the same manner of above 2. As a result, oxygenation proceeded with respect to these aggregated peptides, suggesting that compound 2 recognizes the cross β-sheet structure and reacts.

### 4. Analysis of Excited State of Compound 2

Next, the mechanism of compound 2 in the excited state was investigated. The results of measuring the change in the fluorescence spectrum of compound 2 in the presence and absence of aggregated Aβ are shown in FIG. 5 (A). As a result, it was clarified that Compound 2 fluoresces only in the presence of aggregated Aβ. Furthermore, it was confirmed that singlet oxygen is also produced only in the presence of aggregated Aβ. These results suggest that the binding of compound 2 to aggregated Aβ promotes the fluorescence emission and production of singlet oxygen via intersystem crossing.

In order to investigate the structural cause of such behavior, the structure of compound 2 in the ground state and excited state was optimized using the density functional theory (DFT calculation). As shown in FIG. 5 (B), the dihedral angle of C-N-N-C of the azobenzene structure has a relatively high flatness of 164o in the ground state, whereas the dihedral angle is slightly bent at 149o in the excited state. From the above results, it is considered that this bent structure is involved in the relaxation of the excited state of compound 2 in the solution, and the movement of compound 2 is suppressed by binding to aggregated Aβ, which expressed the oxygenation activity in Aβ selective manner.

### 5. Identification of Oxygenation Site

Glu-C (manufactured by Sigma, 1/50 of the volume) dissolved in water was added to the oxygenation reaction mixture of the above, incubated at 37° C. for 12 to 16 hours, and oxygenation sites in Aβ were determined using LC/MS/MS. As shown in FIG. 6, it was confirmed that oxygenation occurred at the positions of the histidine residue and the methionine residue.

### 6. Examination of Aβ Aggregation Inhibitory Effect

A thioflavin-T assay was performed to verify the inhibitory effect of compound 2 on Aβ aggregation. For the assay, reaction mixture (10 μL) (20 μM Aβ species, 2 μM compound 2, 0.1 M phosphate) was added to 1.25 μM thioflavin-T solution (400 μL) as described in the references above. The thioflavin-T solution used was prepared by adding a 50 μM thioflavin-T aqueous solution (10 μL, ThT purchased from Sigma-Aldrich, Inc.) to a 50 mM glycine NaOH buffer (396 μL, pH 8.5). The fluorescence intensity of the solution (400 μL) was measured at room temperature with an excitation wavelength of 440 nm and an emission wavelength of 480 nm. The fluorescence intensity was measured with a spectral fluorometer RF-5300PC (Shimadzu Corporation) using a rectangular quartz cell (3 mm optical path length). As a fluorescence standard, 50 μM calcein (400 μL) in 100 mM phosphate buffer (pH 7.4) (Purchased calcein from the Institute of DOJIN Chemistry) was used.

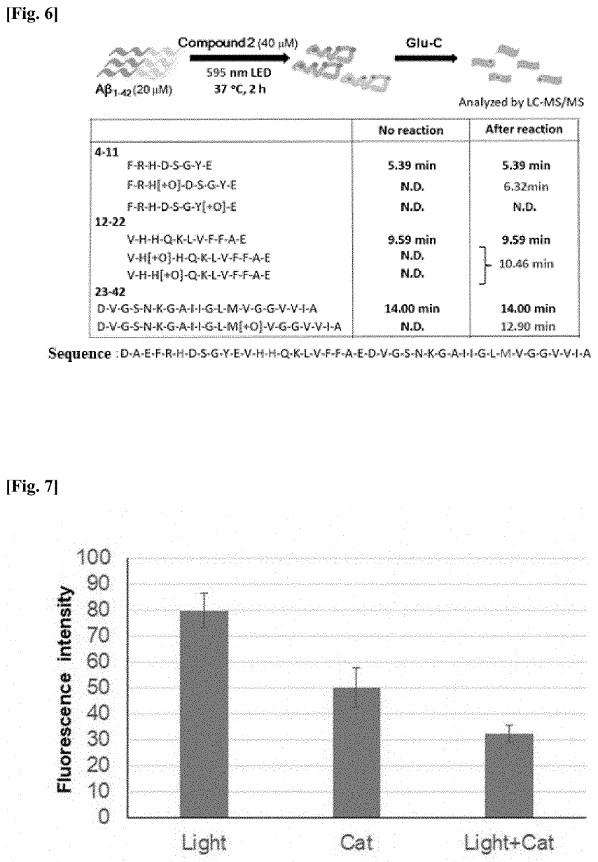

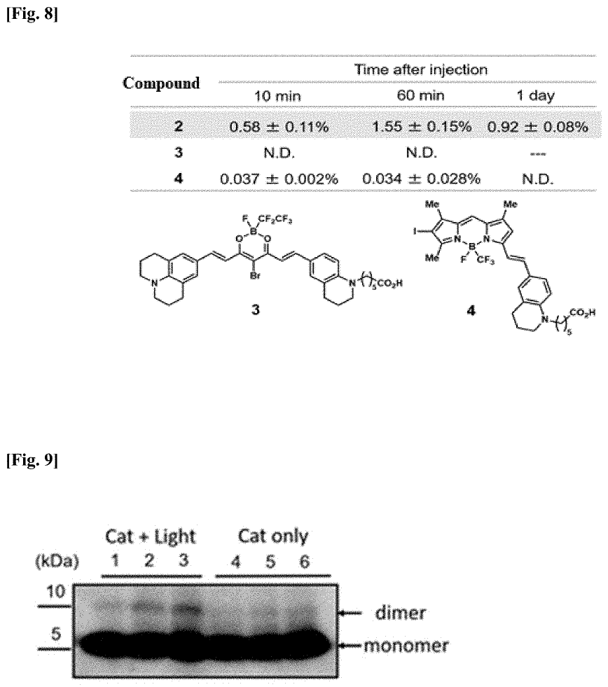

As a result, as shown in FIG. 7, suppression of Aβ aggregation was confirmed by the photooxygenation reaction of Compound 2 with light irradiation. In addition, “Cat” in FIG. 7 means compound 2, and “Light” means that it was irradiated with light.

### 7. Blood-Brain Barrier (BBB) Permeability

Using mice, the abundance in the brain after intravenous administration of the catalyst was evaluated, and the BBB permeability of compound 2 was measured. Further, as a comparative example, the same measurement was performed for compounds 3 and 4, which are conventional photooxygenation catalysts. Compound 3 is a compound disclosed in Non-Patent Document 2 (Ni, J. et al., Chem 2018, 4, 807-820), and compound 4 is Non-Patent Document 3 (Suzuki, T. et al), Chem. Commun. 2019, 55, 6165).

As a result, as shown in FIG. 8, the compound 2 of the present invention showed a sufficiently high BBB permeability as compared with the conventional compounds 3 and 4, and 1.55% of the compound 2 administered was recovered from the brain 60 minutes after the administration.

### 8. Photooxygenation Reaction In Vivo

The oxygenation activity of compound 2 was evaluated in a scheme in which compound 2 was administered to Alzheimer's disease model mice and irradiated with light from outside the body. Compound 2 (1.0 mM, 0.2 mL, 10% DMSO, 15% Kolliphor EL and 75% 1*PBS) was intravenously administered in 11-month-old App knock-in (AppNL-GF/NL-GF) mice expressing human Arctic Aβ. After 60 minutes, the mice were irradiated with 600 nm LED light for 10 minutes. This operation was repeated 5 times in 5 days, and the hippocampus derived from mice were evaluated by Western blotting.

The results are shown in FIG. 9. As a result, in mice treated with catalyst and irradiated with light (lanes 1 to 3), dimers considered to be products due to oxygenation were strongly detected as compared with mice treated with catalyst alone (lanes 4 to 6). This result indicates that compound 2 promotes photooxygenation of Aβ in an in vivo system.

1. A compound represented by the following formula (Ia) or (Ib) or salt thereof: 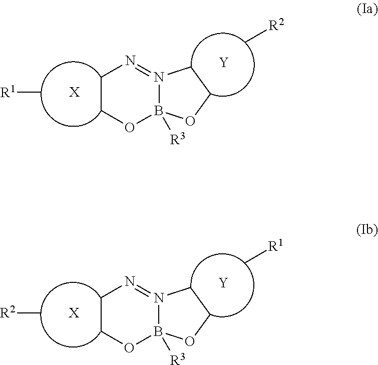
(wherein X and Y are aromatic rings that may be independently the same or different; R¹ is a substituent selected from the group consisting of an amino group, an alkyl group, an alkoxy group, a sulfo group, a phosphoric acid group, and a heteroaryl groups with a hydrophilic substituent, which may be optionally substituted, and which may be present at any position on X or Y; R² is a halogen atom, a selenium atom, or a haloalkyl group having 1-3 carbon atoms, which may be present at any position on Y or X; and R³ is a halogen atom or a haloalkyl group having 1-3 carbon atoms).

2. The compound or salt thereof according to claim 1, wherein X and Y are benzene rings or naphthalene rings.

3. The compound or salt thereof according to claim 1, which is represented by the following formula (IIa) or (IIb): 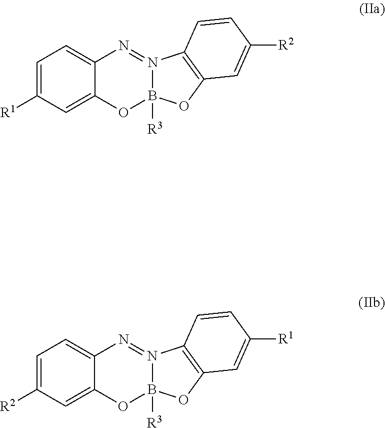
(wherein R¹, R², and R³ are the same as the definition in claim 1).

4. The compound or salt thereof according to any one of claims 1 to 3, wherein R² is a bromine atom, an iodine atom, or a selenium atom.

5. The compound or salt thereof according to any one of claims 1 to 4, wherein R³ is a fluorine atom or a fluoroalkyl group having 1 to 3 carbon atoms.

6. The compound or salt thereof according to any one of claims 1 to 5, wherein R¹ is represented by the following formula (a). 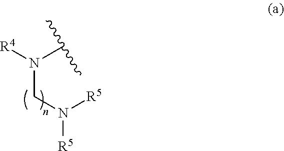
(In the formula, the broken line indicates the connection to X or Y; R⁴ is a hydrogen atom, an alkyl group or an aromatic ring which may be optionally substituted; R⁵ is a hydrogen atom, an alkyl group or an aromatic ring, which may be optionally substituted, which may be independently the same or different; n is an integer of 0 to 5.).

7. A compound selected from the following groups or salt thereof: 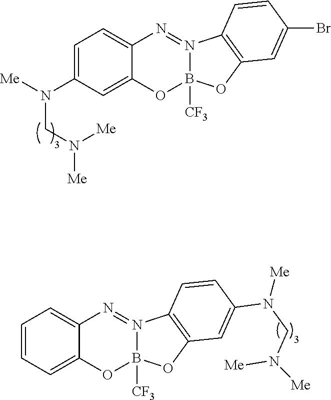
(wherein “Me” represents a methyl group.).

8. An oxygenation catalyst for pathogenic amyloids comprising the compound according to any one of claims 1 to 7 or salt thereof.

9. An agent for suppressing the aggregation of pathogenic amyloids, which comprises the compound according to any one of claims 1 to 7 or salt thereof.

10. A pharmaceutical composition comprising the compound according to any one of claims 1 to 7 or salt thereof, and a pharmaceutically acceptable carrier.

11. The pharmaceutical composition according to claim 10, which is a prophylactic or therapeutic agent for a disease associated with pathogenic amyloids.

12. The pharmaceutical composition according to claim 11, wherein the disease associated with the pathogenic amyloids is Alzheimer's disease.

13. Use of the compound according to any one of claims 1 to 7 or salt thereof in producing an agent for prevention or treatment of a disease associated with pathogenic amyloids.

14. A method for preventing or treating a disease associated with pathogenic amyloids, which comprises administering an effective amount of the compound according to any one of claims 1 to 7 or salt thereof.

15. The method according to claim 14, comprising irradiating the affected area of the patient with light from outside the body after administration of the compound according to any one of claims 1 to 7 or salt thereof.

16. The method according to claim 14 or 15, wherein the disease associated with the pathogenic amyloids is Alzheimer's disease.
